(19) United States
(12) Patent Application Publication
(10) Pub. No.: US 2014/0120590 A1
(43) Pub. Date: May 1, 2014

(54) NOVEL STRAIN OF MICROALGAE OF THE ODONTELLA GENUS FOR THE PRODUCTION OF EPA AND DHA IN MIXOTROPHIC CULTIVATION MODE

(71) Applicants: Pierre Calleja, Bordeaux (FR); Khadidja Romari, Clermont-Ferrand (FR)

(72) Inventors: Pierre Calleja, Bordeaux (FR); Khadidja Romari, Clermont-Ferrand (FR)

(73) Assignee: FERMENTALG, Libourn (FR)

(21) Appl. No.: 14/124,367

(22) PCT Filed: Jun. 7, 2012
(86) PCT No.: PCT/FR2012/051282

§ 371 Date: Dec. 6, 2013

(30) Foreign Application Priority Data
Jun. 8, 2011 (FR) 1154981

(51) Int. Cl. C12P 7/64; C12R 1/89

(52) U.S. Cl. CPC C12P 7/6427; C12R 1/89
USPC 435/134; 435/257.1

(57) ABSTRACT

A novel strain of microalga of the Odontella genus, capable of growing in mixotrophic mode, and a method for selecting and culturing such a strain allow production of polyunsaturated fatty acids, notably EPA and DHA, in heterotrophic or mixotrophic mode.

The invention relates to a novel strain of a microalga of the Odontella genus, capable of growing under heterotrophic and mixotrophic conditions, and to a method for selecting and culturing said microalgae, allowing production of polyunsaturated fatty acids, notably EPA and DHA, in heterotrophic or mixotrophic mode.

This novel strain is particularly useful for producing EPA and DHA, notably in a culture method performed in mixotrophic mode, wherein the supply of light is in the form of flashes.

### PREAMBLE

It is known that microalgae are photosynthetic microorganisms with an autotrophic character, i.e. they have the capacity to grow autonomously by photosynthesis.

Microalgae develop both in marine aquatic media and in fresh or brackish waters, as well as in various land habitats.

Most of the microalgae species encountered in fresh water or in oceans are strictly autotrophic, i.e. they can only grow by photosynthesis.

However, a certain number of microalgae species of very varied families and origins, are found to be not strictly autotrophic. Thus, some of them, said to be heterotrophic, are capable of developing in the total absence of light, by fermentation, i.e. by using organic matter.

Other microalgae species, for which photosynthesis remains essential for their development, are capable of benefiting both from photosynthesis and from the organic matter present in their environment. These intermediate species, said to be mixotrophic, can be cultured in the presence of both light and organic matter.

This particularity of so-called [symbol] mixotrophic [symbol] microalgae seems to be related to their metabolism, which allows them to carry out photosynthesis and fermentation simultaneously. Both types of metabolism coexist with a positive overall effect on the growth of said microalgae [Yang C. et al. (2000) Biochemical Engineering Journal 6:87-102].

At present, the classification of microalgae is still widely based on morphological criteria and on the character of the photosynthetic pigments which their cells contain. Consequently, it is not very indicative of the autotrophic, heterotrophic or mixotrophic character of the different species of algae, whereas the latter cover a very great diversity of species and of forms [Dubinsky et al. 2010, Hydrobiologia, 639:153-171].

Microalgae are currently the subject of many industrial projects since certain species are capable of accumulating or secreting significant amounts of lipids, notably, polyunsaturated fatty acids.

Among these polyunsaturated fatty acids, certain highly unsaturated acids from the series of Omega-3s (PUFA-ω3), in particular eicosapentaenoic acid (EPA, C20:5 ω3) and docosahexaenoic acid (DHA, C22:6 ω3) have a recognized nutritional importance and have strong potential in terms of therapeutic applications [Horrocks L. A. et al. (2000) Health Benefits of DHA. Pharmacol. Res. 40: 211-225].

Fish oils from the fish industry are presently the main commercial source of these types of fatty acids. However, while these oils find new applications (food supplement in aquaculture, incorporation into margarines), marine halieutical resources are becoming scarce because of intensive fishing activity.

New sources of EPA and DHA, therefore, have to be sought in order to meet, in the future, the increasing demand from the market for these types of polyunsaturated fatty acids.

In addition to their capability of synthesizing fatty acids de novo, microalgae provide several advantages compared with fish oils. In particular, they may be cultured in vitro under controlled conditions, which allows production of a biomass with a relatively constant biochemical composition. On the other hand, unlike fish oils, the lipids from microalgae do not have any unpleasant smell and contain little or no cholesterol. Finally, the lipids produced by microalgae have a generally simpler fatty acid profile than that of fish oils, which limits the steps for separating the fatty acids of interest.

The main EPA- and DHA-producing microalgae are marine species belonging to various phyla. However, from the hundreds of thousands of species covered by these phyla, only a small number of species away from each other in a taxonomic rank, have a high content of EPA and DHA. Among the species capable of producing significant quantities of EPA and/or of DHA, the most often mentioned are those belonging to the genera Schizotrium sp., Crypthecodinium sp. (Dinophyceae), Phaeodactylum sp. (Bacillariophyceae, Naviculales) and Odontella sp. (Bacillariophyceae, Coscinodiscophyceae).

The microalgae of the Odontella genus are unicellular algae of large size, the length of which may attain 35 to 50 microns. They have a silica frustule consisting of two symmetrical valves. These are ubiquitous and cosmopolitan microalgae of the neritic zone, not forming dense plankton populations and which are often found associated with diverse species of benthic macroalgae in coastal zones. In the natural state, the species Odontella aurita accumulates between 1.6% and 3.4% by total dry weight of EPA, which represents on average 21% of the total fatty acids produced by this microalga.

Odontella aurita is generally cultured in autotrophic mode in external pools for its use in animal food, notably for feeding larvae of fish and crustaceans [Pulz et Gross (2004) Valuable products from biotechnology of microalgae, Appl. Microbiol. Biotechnol. 65(6):635-648].

Nevertheless, the culture of microalgae in autotrophic mode in open pools is not very adapted to industrial utilization of the latter. Indeed, in the perspective of an intensive utilization of microalgae, the biomass production has to be achieved in a large amount, in closed, large photo-bioreactors. However, it is difficult under such conditions to provide satisfactory illumination to the whole of the cells contained in the culture medium, in particular when the density of the microalgae becomes significant.

An alternative to the autotrophic cultures of microalgae of the Odontella genus would be to practice heterotrophic cultures, i.e. in the absence of light, with provision of energy in the form of carbon-containing substrates, or else, mixotrophic cultures, i.e. in the presence of a supply of light of lower intensity, and with a supply of organic substrate.

However, up to now, the microalgae of the Odontella genus have not been able to be cultured under such conditions.

It is therefore unexpectedly that the applicant has managed to isolate a microalga strain of the Odontella genus, which may be cultured in heterotrophic and mixotrophic mode, capable of producing DHA and EPA in a satisfactory amount.

This novel strain of Odontella was isolated from the environment and cultured by the inventor, and more particularly according to a method developed by the latter, consisting of culturing the microalgae under mixotrophic conditions, in the presence of discontinuous illumination, notably, in the form of flashes.

The close alternation of illuminated phases and dark phases, generally perceived as stressful for microalgae, surprisingly made it possible to obtain a high production of polyunsaturated fatty acids from this strain. The application of such a strain, according to the invention, opens the perspective of industrial production of polyunsaturated fatty acids in fermenters benefiting from reduced light supply, and should therefore make possible savings in energy and surface area, compared with existing autotrophic culture modes.

The strain of Odontella, FCC 675, which is the first of these strains to have been selected according to the present invention, was deposited according to the provisions of the Treaty of Budapest at the CCAP (Culture Collection of Algae and Protozoa, Scottish Association for Marine Science, Dunstaffnage Marine Laboratory, Oban, Argyll PA371QA, Scotland, United Kingdom) on May 27, 2011 and was assigned the accession number CCAP 1054/5.

The various aspects and advantages of the invention are detailed hereafter.

### DETAILED DESCRIPTION

The present invention therefore relates to an isolated microalga of the Odontella genus (Phylum: Bacillariophyta, Class: Coscinodiscophyceae, Family: Eupodicaceae) [ITIS, Catalogue of Life, 2010], characterized in that it may be cultured in heterotrophic or mixotrophic mode.

The fact that this microalga may be cultured in heterotrophic mode is appreciated by its capability of multiplying in darkness in a standard culture medium, usually used for culturing Odontella in autotrophic mode, preferably a mineral medium, in which a carbon-containing substrate is added. By mineral medium, is meant a culture medium consisting in an aqueous solution, often based on sea water, in which are dissolved inorganic compounds, as well as, optionally, vitamins and certain amino acids. A suitable mineral medium for the culture of Odontella is for example the f/2 medium [Guillard, R. R. L. (1975) Culture of phytoplankton for feeding marine invertebrates. pp 26-60. In Smith W. L. and Chanley M. H (Eds.) Culture of Marine Invertebrate Animals. Plenum Press, New York, USA; Guillard, R. R. L. and Ryther, J. H. (1962) Studies of marine planktonic diatoms. I. Cyclotella nana Hustedt and Detonula confervacea Cleve. Can. J. Microbiol. 8: 229-239].

The fact that this microalga may be cultured in mixotrophic mode is appreciated by the capability of the microalga of multiplying in the presence of a light supply, in a culture medium similar to the one described above, i.e. a medium usually used for culturing Odontella in autotrophic mode, but into which a carbon-containing substrate is added.

Generally, the intensity of the light supply is greater than or equal to 5 μE, preferentially, comprised between 5 and 300 μE, more preferentially, between 10 and 200 μE, and even more preferentially, between 20 and 150 μE.

In heterotrophy or mixotrophy, a carbon-containing substrate is brought into the culture. The carbon-containing substrate comprises or consists in, in pure form or as a mixture, generally, one or several of the following compounds: starch, glucose, xylose, arabinose, lactose, lactate, cellulose and derivatives thereof, saccharose, acetate and/or glycerol.

Products from the biotransformation of starch, for example, from maize, wheat, or potato, notably, starch hydrolyzates, which consist of small sized molecules, may form carbon-containing substrates which may be used for culturing microalgae in heterotrophic or mixotrophic mode.

Culture in mixotrophic or heterotrophic mode of this microalga is preferentially carried out in the presence of at least 5 mM, preferably, at least 10 mM, more preferentially, at least 20 mM and still more preferentially, more than 50 mM of a carbon-containing substrate. One skilled in the art knows how to determine the maximum concentrations of the carbon-containing substrate to be used. Generally, culture in mixotrophic or heterotrophic mode of this microalga may be carried out in the presence of 10-200 mM, preferably, between 20 and 50 mM of carbon-containing substrate.

A continual substrate supply is ensured during the culture, in order to allow the cells to accumulate a significant concentration of lipids. Additional substrate is added to the culture medium during the culture method so as to maintain a constant concentration. One skilled in the art knows how to determine the amounts of carbon-containing substrate to be added to the culture in order to maintain a constant concentration of the carbon-containing substrate in the culture medium. Generally, the culture may thus be carried out with cumulated concentrations of carbon-containing substrate from 5 mM to 1 M, preferably, from 50 mM to 800 mM, more preferentially, from 70 mM to 600 mM and even more preferentially, from 100 mM to 500 mM.

For the purposes of the present invention, arabinose and xylose are the preferred carbon-containing substrates for culturing Odontella in heterotrophic mode according to the invention.

Acetate and saccharose are the preferred carbon-containing substrates for culturing Odontella in mixotrophic mode according to the invention.

The invention thus relates to a method for culturing or selecting a microalga of the Odontella genus with a heterotrophic or mixotrophic character according to the invention, characterized in that it comprises the following steps:
culturing one or several strains of the Odontella genus in darkness or in the presence of a light supply;
maintaining said culture over several generations in the presence of a carbon-containing substrate, as described earlier, in said culture medium;
harvesting the thus obtained Odontella cells.

The culture method according to the invention notably relates to the production of polyunsaturated fatty acids, more particular EPA and DHA, which accumulate in the thus selected or produced microalgae.

Such a culture method proves to be particularly advantageous when a variable or discontinuous light supply is applied, in other words when the light flux provided to the cultured algae is variable or discontinuous over time.

Contrary to common beliefs, it appeared that variable or discontinuous illumination of the cultures, in particular, when used in culture in mixotrophic mode, had a favorable impact on the development of algae and made it possible to increase their productivity, notably as far as their lipid production is concerned.

Without being bound by theory, the inventor believes that a discontinuous or variable light supply to the microalgae has the effect of causing a [symbol] stress [symbol] favorable to the synthesis of lipids. This phenomenon may be partly explained by the fact that, in nature, microalgae tend to accumulate lipid reserves to withstand the constraints of their environment.

By discontinuous illumination, it is meant illumination punctuated with periods of darkness. The periods of darkness may be more than one quarter of the time, preferably, half or more of the time, during which the algae are cultured.

According to a preferred aspect of the invention, the illumination is discontinuous and, more preferentially, in the form of flashes, i.e. over periods of short durations. The successive illumination phases are then generally comprised between 5 seconds and 10 minutes, preferably, between 10 seconds and 2 minutes, more preferentially, between 20 seconds and 1 minute.

According to another embodiment of the invention, the illumination may be variable, which means that the illumination is not interrupted by phases of darkness, and the light intensity varies over time. This light variation may be periodical, cyclic or even random.

According to the invention, the illumination may vary continuously, i.e. the light intensity is not constant and permanently varies over time (dμmol (photons)/dt≠0).

According to the invention, it is also possible to have a light supply combining continuous and discontinuous illumination phases.

The invention is, in particular, directed to a method for culturing microalgae of the Odontella genus, characterized in that said algae are cultured in darkness with a discontinuous or variable supply of light over time, the intensity of which, in micromoles of photons, varies by an amplitude equal to or greater than 10 μmol.m⁻².s⁻¹, preferably, equal to or greater than 50 μmol.m⁻².s⁻¹, more preferentially, equal to or greater than 100 μmol.m⁻².s⁻¹, at a rate of one or several times per hour, advantageously, more than once per hour. The common point of these different discontinuous or variable illumination modes lies in the fact that, according to the invention, the intensity of the light brought to the algae in culture, expressed in micromoles of photons per second per meter square (μmol.m⁻².s⁻¹), varies at least once within the same hour. The amplitude of this variation of the light intensity variation is generally greater than 10 μmol.m⁻².s⁻¹, preferentially, greater than or equal to 20 μmol.m⁻².s⁻¹, more preferentially, greater than or equal to 50 μmol. m⁻². s⁻¹. In other words, every hour, preferably several times within the hour, the light intensity attains a high value and a low value, the difference between these values being equal to or greater than that indicated above. Preferably, said light intensity successively attains the values 50 μmol. m².s⁻¹ and 100 μmol.m⁻².s⁻¹ every hour, more preferentially, the values 0 and 50 μmol. m².s⁻¹, still more preferentially, the value 0 and 100 μmol. m².s⁻¹.

It is known that 1 μmol. m².s⁻¹ corresponds to 1 μE m⁻².s⁻¹ (Einstein), a unit often used in the literature.

The light supply in the cultures may be obtained by lamps distributed around the external wall of the fermenters. A clock triggers these lamps for defined illumination periods. The fermenters are preferentially located in a temperature-controlled enclosure, shielded from daylight.

According to an embodiment of the invention, the cultures may be obtained in a fermenter in which the culture medium circulates regularly to reach an illuminated part of the fermenter. Such a fermenter may, for example, may be a device with a circular pipe, a portion of which is transparent and illuminated from the outside. The culture medium and the suspended algae, while actively circulating through the illuminated portion of such a device, are thus periodically in contact with light.

A particular strain of Odontella, FCC 675, isolated, selected and cultured by the applicant, was deposited at the CCAP on May 27, 2011 under accession number CCAP 1054/5. According to ongoing taxonomic analyses, the latter belongs to the species Odontella aurita. Nevertheless, taking into account its filiation with other Odontella species, the invention relates to any microalga species of the Odontella genus having a mixotrophic character, as described in the present application.

As the applicant could observe, the fact that the strains selected according to the method of the invention have good growth capabilities in heterotrophic or mixotrophic mode, predisposes these strains to higher production of polyunsaturated fatty acids, notably EPA and DHA.

The culture method according to the invention thus allows selection of Odontella strains with a mixotrophic character, similar to the strain isolated by the applicant and deposited at the CCAP, having a high yield of polyunsaturated fatty acids.

To screen the strains, various Odontella strains may be cultured in parallel on microplates, in the same enclosure, with a precise monitoring of the conditions and of the development of the various cultures. It is, thus, easy to determine the response of the various strains to discontinuous illumination and, if necessary, upon adding one or several carbon-containing substrates into the culture medium. The strains which react favorably to the discontinuous illumination and to the carbon-containing substrates, generally provide a better yield for the production of lipids in terms of quality (polyunsaturated fatty acids more abundant in the lipid profile) and in terms of quantity (the lipids contain a higher proportion of EPA and of DHA).

The microalgae may be selected in a fermenter from a diversified pool of microalgae, and from which one aims to select the variants advantaged by this selection mode according to the invention, combining discontinuous or variable light with mixotrophic culture conditions. In this case, the culture is carried out by maintaining the microalgae in cultures over many generations, and then isolation of the components which have become a majority in the culture medium, is performed at the end of the culture.

The culture method according to the invention is more particularly characterized in that the culture of the strains is carried out over several generations, preferably in mixotrophic mode, and, in that the cells loaded with lipids are harvested.

The invention also relates to a method for enriching microalgae of the Odontella genus in polyunsaturated fatty acids, characterized in that it comprises the selection and culture of microalgae of the Odontella genus, in mixotrophic or heterotrophic mode, more particularly according to the culture methods described earlier.

The invention also relates to the production of lipids, notably polyunsaturated fatty acids, via the culture of microalgae of the Odontella genus with a heterotrophic or mixotrophic character, and then, the recovery of the thus cultured microalgae to extract therefrom the lipid content, in particular, EPA and/or DHA. These microalgae are preferably cultured or selected according to the methods mentioned earlier.

The culture of Odontella strains according to the invention in mixotrophic or heterotrophic mode generally allows an increase in the total biomass by more than 20%, most often by more than 30% and even sometimes by more than 40% as compared with the culture of a same strain of Odontella in autotrophic mode.

Moreover, the content of total EPA and DHA lipids in the total lipids extracted from the thus cultured Odontella strains, represents more than 10%, generally more than 30%, very often more than 40%, or even more than 50% of the total cell lipids by dry weight.

The methods for selectively extracting EPA and DHA are known to one skilled in the art and are, for example, described by Bligh, E. G. and Dyer, W. J. [A rapid method of total lipid extraction and purification (1959) Can. J. Biochem. Physiol., 37: 911-917]. The EPA and DHA which are thus extracted may be used as additives in nutritional compositions, such as formula milk, or else, in cosmetic or therapeutic compositions.

The microalgae, selected, cultured or enriched in polyunsaturated fatty acids according to the method of the invention may be used directly in the hydrated or dehydrated form, or after transformation, as a nutritional supplement foodstuff, notably, in fish farming, or as an ingredient used in the composition of cosmetic or therapeutic products.

### EXAMPLE

Culture of Odontella aurita Strains in a Bioreactor

The cultures are grown in 2L usable capacity fermenters (bioreactors) with dedicated automatic equipment with computerized supervision. The pH of the system is adjusted by adding base (a 1N sodium hydroxide solution) and/or acid (1N sulfuric acid solution). The culture temperature is set to 23° C. Stirring is achieved using 3 stirring rotors placed on the shaft according to the Rushton configuration (3-blade propellers with down pumping). The stirring rate and the aeration flow rate are regulated to a minimum of 100 rpm and to a maximum of 250 rpm and Qₘᵢₙ=0.5 vvm/Qₘₐₓ=2 vvm respectively. The bioreactor is equipped with an external lighting system surrounding the transparent tank. The intensity and the light cycles are controlled by dedicated automatic equipment with computerized supervision.

The reactors are inoculated with a preculture prepared on a mixing table (140 rpm) in a controlled-temperature enclosure (22° C.) and illuminated continuously at 100 μEm⁻²s⁻¹. Precultures and cultures are prepared in bioreactors in f/2 medium. The organic carbon used for the mixotrophic culture in a bioreactor is sodium acetate at concentrations of between 20 mM and 50 mM. The carbon-containing organic substrate is added in the culture medium in >fed-batch≦ mode. The heterotrophic culture conditions are identical to those of mixotrophy in the absence of light.

### Monitoring of cultures

The total biomass concentration is monitored by measuring the dry mass (filtration on a Whatman GFC filter, and then oven drying in vacuo, 65° C. and −0.8 bars, for a minimum of 24 h before weighing).

Regarding the quantification of the total number of lipids, 10⁷ cells/mL were extracted. Methods for extracting lipids are known to one skilled in the art and are, for example, described by Bligh, E. G. and Dyer, W. J. [A rapid method of total lipid extraction and purification (1959) Can. J.Biochem. Physiol 37:911-917].

### Flashing Light

The light supply in the bioreactor cultures was obtained by LED lamps distributed around the external wall of the fermenters. A clock triggers these LEDs for illumination times or pulses between 10 and 100 μE. The light intensity of the flash system used in mixotrophy is the same as the one used in autotrophy (control).

| | | Mixotrophy |
|---|---|---|
| Strain Odontella | Heterotrophy | with flashes |
| Biomass | >150% | >200% |
| (% relative to | | |
| autotrophy) | | |
| Total lipids | >20% | >35% |
| (% relative to | | |
| autotrophy) | | |
| EPA and DHA | >25% | >30% |
| (% relative to | | |
| autotrophy) | | |

1-12. (canceled)

13. Microalga corresponding to the Odontella strain deposited at the CCAP on May 27, 2011 under accession number CCAP 1054/5.

14. Method for culture or selection of a microalga of the Odontella genus, characterized in that it comprises the following steps:
culturing one or several strains of the Odontella genus in darkness or in the presence of a light supply;
maintaining said culture over several generations in the presence of a carbon-containing substrate comprising at least 20 mM acetate, glucose, xylose, arabinose, lactose, saccharose, acetate or glycerol in the culture medium;
harvesting the thus obtained Odontella cells.

15. Culture method according to claim 14, wherein the carbon-containing substrate comprises acetate or saccharose.

16. Culture method according to claim 14, wherein the microalga strain of the Odontella genus is the Odontella strain deposited at the CCAP on May 27, 2011 under accession number CCAP 1054/5.

17. Culture method according to claim 14, characterized in that culture of the strains is carried out in mixotrophic mode with a light supply, the intensity of which is comprised between 5 and 300 μE, preferably, between 10 and 200 μE, more preferentially, between 20 and 150 μE.

18. Culture method according to claim 17, characterized in that said carbon-containing substrate comprises arabinose or xylose.

19. Culture method according to claim 14, characterized in that culture of the strains is carried out in mixotrophic mode with a discontinuous or variable supply of light over time, the intensity of which, in micromoles of photons, varies by an amplitude of more than 50 μmol.m⁻².s⁻¹ at a rate of at least once per hour.

20. Culture method according to claim 14, characterized in that the supply of light is in the form of flashes.

21. Culture method according to claim 20, characterized in that flashing consists of successive illumination phases with a duration comprised between 5 seconds and 10 minutes, preferably, between 10 seconds and 2 minutes, more preferentially, between 20 seconds and 1 minute.

22. Method for enriching microalgae of the Odontella genus in EPA (eicosapentaenoic acid) and/or DHA (docosahexaenoic acid), characterized in that it comprises the culture of at least one microalga strain of the Odontella genus in heterotrophic or mixotrophic mode, according to the culture method of claim 14.

23. Microalga of the Odontella genus which may be obtained according to the method of claim 14, characterized in that its EPA and DHA content represents more than 40% and, more preferentially, more than 50% of the total cell lipids.

24. EPA or DHA production method, characterized in that EPA and/or DHA are extracted from the lipid content of microalgae obtained according to the method of claim 14.

25. Culture method according to claim 15, wherein the microalga strain of the Odontella genus is the Odontella strain deposited at the CCAP on May 27, 2011 under accession number CCAP 1054/5.

26. Culture method according to claim 15, characterized in that culture of the strains is carried out in mixotrophic mode with a light supply, the intensity of which is comprised between 5 and 300 μE, preferably, between 10 and 200 μE, more preferentially, between 20 and 150 μE.

27. Culture method according to claim 15, characterized in that culture of the strains is carried out in mixotrophic mode with a discontinuous or variable supply of light over time, the intensity of which, in micromoles of photons, varies by an amplitude of more than 50 μmol.m⁻².s⁻¹ at a rate of at least once per hour.

28. Culture method according to claim 15, characterized in that the supply of light is in the form of flashes.
